(19) United States
(12) Patent Application Publication
(10) Pub. No.: US 2013/0245047 A1
(43) Pub. Date: Sep. 19, 2013

(54) COMPOSITIONS FOR TREATING CANCER-RELATED FATIGUE AND METHODS OF SCREENING THEREOF

(71) Applicant: TENERA THERAPEUTICS, LLC, Boston, MA (US)

(72) Inventors: Stephen T. SONIS, Wayland, MA (US); Edward G. FEY, Boston, MA (US); Edward B. RUBENSTEIN, Houston, TX (US)

(73) Assignee: Tenera Therapeutics, LLC, Boston, MA (US)

(21) Appl. No.: 13/886,035

(22) Filed: May 2, 2013

Related U.S. Application Data
(63) Continuation of application No. 13/231,098, filed on Sep. 13, 2011, now abandoned.

(51) Int. Cl. A61K 31/522

(52) U.S. Cl. CPC A61K 31/522
USPC 514/263.36; 544/271

(57) ABSTRACT

An animal model has been developed based the understanding that a central mechanism in patients with CTRF is that chemotherapy and/or radiation initiates canonical pathways leading to the development of disrupted sleep architecture, resulting in disruption of REM sleep and fatigue and cognitive dysfunction. Drugs that restore the activity patterns and levels towards normal and/or decrease the pro-inflammatory cytokines associated with the disrupted sleep, should be effective in alleviating one or more symptoms of CTRF. Pentoxifylline was demonstrated to improve activity levels in animals treated with etoposide.

### CROSS-REFERENCE TO RELATED APPLICATIONS

This application is a continuation of U.S. patent application Ser. No. 13/231,098 filed on Sep. 13, 2011, which claims benefit of and priority to U.S. Provisional Patent Application No. 61/382,269 filed on Sep. 13, 2010, both of which are incorporated by reference herein in their entirety.

### FIELD OF THE INVENTION

The present invention is generally in the field of compounds for treatment of one or more symptoms of cancer-related fatigue and methods of screening for compounds for treatment of cancer-related fatigue.

### BACKGROUND OF THE INVENTION

Fatigue occurs in 14% to 96% of people with cancer, especially those undergoing treatment for their cancer. Fatigue is complex, and has biological, psychological, and behavioral causes. Fatigue is difficult to describe and people with cancer may express it in different ways, such as saying they feel tired, weak, exhausted, weary, worn-out, heavy, or slow. Health professionals may use terms such as asthenia, fatigue, lassitude, prostration, exercise intolerance, lack of energy, and weakness to describe fatigue.

Fatigue can be described as a condition that causes distress and decreased ability to function due to a lack of energy. Specific symptoms may be physical, psychological, or emotional. To be treated effectively, fatigue related to cancer and cancer treatment needs to be distinguished from other kinds of fatigue.

Fatigue may be acute or chronic. Acute fatigue is normal tiredness with occasional symptoms that begin quickly and last for a short time. Rest may alleviate fatigue and allow a return to a normal level of functioning in a healthy individual. Chronic fatigue syndrome (“CFS”) describes prolonged debilitating fatigue that may persist or relapse, and is not related to cancer. Fatigue related to cancer, also referred to as cancer treatment-related fatigue (“CTRF”) is called chronic because it lasts over a period of time and is not completely relieved by sleep and rest. Chronic fatigue diagnosed in patients with cancer may be called “cancer fatigue” or “cancer-related fatigue”. Although many treatment- and disease-related factors may cause fatigue, the exact process of fatigue in people with cancer is not known.

Fatigue is the most common side effect of cancer treatment with chemotherapy, radiation therapy, or selected biologic response modifiers. Cancer treatment-related fatigue generally improves after treatment is completed, but some level of fatigue may persist for months or years following treatment. Fatigue is also seen as a presenting symptom in cancers that produce problems such as anemia, endocrine changes, and respiratory obstruction and is common in people with advanced cancer who are not receiving active cancer treatment. Most of the research on fatigue in people with cancer has been conducted on people actively undergoing cancer treatment, with a few studies focused on people receiving palliative care for terminal cancer and some research on people after treatment is completed. Cancer treatment-related fatigue is reported in 14% to 96% of people undergoing cancer treatment (Fossa et al., J Clin Oncol 21 (7): 1249-54, 2003; Miaskowski et al. Principles and Practice of Supportive Oncology Updates 1 (2): 1-10, 1998; Irvine et al. Cancer Nurs 14 (4): 188-99, 1991; Vogelzang et al., The Fatigue Coalition. Semin Hematol 34 (3 Suppl 2): 4-12, 1997; Detmar et al. J Clin Oncol 18 (18): 3295-301, 2000; Costantini et al. Qual Life Res 9 (2): 151-9, 2000; Cella et al. Cancer 94 (2): 528-38, 2002).

The fatigue experienced as a side effect of cancer treatment is differentiated from the fatigue experienced by healthy people in their daily lives. Healthy fatigue is frequently described as acute fatigue that is eventually relieved by sleep and rest; cancer treatment-related fatigue is categorized as chronic fatigue because it is present over a long period of time and is not completely relieved by sleep and rest. The pattern of fatigue associated with cancer treatment varies according to type and schedule of treatment. For example, people treated with cyclic chemotherapy regimens generally exhibit peak fatigue in the days following treatment, then report lower levels of fatigue until the next treatment; however, those receiving external beam radiation therapy report gradually increasing fatigue over the course of treatment of the largest treatment field. Few studies of people receiving cancer treatment have addressed the issue of fatigue as a result of the emotional distress associated with undergoing a diagnostic evaluation for cancer and the effects of medical and surgical procedures used for that evaluation and for initial treatment. Because most adults enter the cancer care system following at least one surgical procedure and because surgery and emotional distress are both associated with fatigue, it is likely that most people beginning nonsurgical treatment are experiencing fatigue at the beginning of treatment.

Fatigue assessment in clinical practice takes many forms, relying mostly on a single-item fatigue intensity rating similar to that used for initial pain assessment. A number of multiple-item tools originally developed for fatigue research have also been used in clinical practice. Most of these tools include symptom dimensions other than fatigue intensity, such as the impact or consequences of fatigue, timing of fatigue, related symptoms, and self-care actions.

Except for chemotherapy-induced anemia, the mechanisms responsible for fatigue in people with cancer are not known. Understanding the causes of fatigue in people with cancer is especially challenging because each individual is likely to experience multiple possible causes of fatigue simultaneously. This multifactorial etiologic hypothesis is apparent in the various models that have been proposed for the study of fatigue (Miaskowski et al. Principles and Practice of Supportive Oncology Updates 1 (2): 1-10, 1998; Morrow et al. Support Care Cancer 10 (5): 389-98, 2002). Energy balance, stress, life demands, sleep, neurophysiologic changes, disruption of circadian rhythms, and neuroimmunological changes are generally incorporated in these models, based on the rationale that these factors are associated with fatigue in contexts other than cancer (Aistars et al Oncol Nurs Forum 14 (6): 25-30, 1987).

The cancer literature supports some of these variables. Sleep disruption was associated with fatigue in women receiving adjuvant chemotherapy for breast cancer. One study demonstrated variations in energy requirements in people with cancer and proinflammatory cytokines are elevated in some studies of people experiencing persistent fatigue following cancer treatment (Kaempfer et al Cancer Nurs 9 (4): 194-199, 1986; Ancoli-Israel et al. Support Care Cancer 14 (3): 201-9, 2006). In addition, concurrent medications such as analgesics, hypnotics, antidepressants, antiemetics, steroids, or anticonvulsants, many of which act on the central nervous system, can significantly compound the problem of fatigue.

The association of fatigue with the major cancer treatment modalities of surgery, chemotherapy, radiation therapy, and biologic response modifier therapy caused speculation that fatigue resulted from tissue damage or accumulation of the products of cell death. Interest in the effects of cancer treatment on the production of proinflammatory cytokines is based on recognition of the strong fatigue-inducing effect of some biologic response modifiers such as interferon alpha and the finding of elevated levels of proinflammatory cytokines in people experiencing persistent fatigue following cancer treatment. Fatigue also has long been associated with radiation exposure. The phenomenon of fatigue accompanying radiation therapy, however, is not well understood. Specific etiologic factors and correlates of fatigue associated with radiation therapy have not been identified.

Fatigue is a dose-limiting toxicity of treatments with a variety of biotherapeutic agents. Biotherapy exposes patients with cancer to exogenous and endogenous cytokines. Biotherapy-related fatigue usually occurs as part of a constellation of symptoms called flulike syndrome. This syndrome includes fatigue, fever, chills, myalgias, headache, and malaise. Mental fatigue and cognitive deficits have also been identified as biotherapy side effects.

Evidence suggests that anemia may be a major factor in cancer-related fatigue (CRF) and quality of life in cancer patients. Anemia can be related to the disease itself or caused by the therapy. Occasionally, anemia is simply a co-occurring medical finding that is related to neither the disease nor the therapy. The impact of anemia varies depending on factors such as the rapidity of onset, patient age, plasma-volume status, and the number and severity of co-morbidities. Fatigue often occurs when the energy requirements of the body exceed the supply of energy sources. In people with cancer, three major mechanisms may be involved: alteration in the body's ability to process nutrients efficiently, increase in the body's energy requirements, and decrease in intake of energy sources.

Numerous factors related to the moods, beliefs, attitudes, and reactions to stressors of people with cancer can also contribute to the development of chronic fatigue. Anxiety and depression are the most common co-morbid psychiatric disorders of cancer-related fatigue. Often, fatigue is the final common pathway for a range of physical and emotional etiologies.

Depression can be a co-morbid, disabling syndrome that affects approximately 15% to 25% of persons with cancer. The presence of depression, as manifested by loss of interest, difficulty concentrating, lethargy, and feelings of hopelessness, can compound the physical causes for fatigue in these individuals and persist long past the time when physical causes have resolved. Anxiety and fear associated with a cancer diagnosis, as well as its impact on the person's physical, psychosocial, and financial well-being, are sources of emotional stress. Distress associated with the diagnosis of cancer alone may trigger fatigue.

Impairment in cognitive functioning, including decreased attention span and impaired perception and thinking, is commonly associated with fatigue. Although fatigue and cognitive impairments are linked, the mechanism underlying this association is unclear. Attention fatigue may be relieved by activities that promote rest and recovery of directed attention. Although sleep is necessary for relieving attention fatigue and restoring attention, it is insufficient when attention demands are high. Disrupted sleep, poor sleep hygiene, decreased nighttime sleep or excessive daytime sleep, and inactivity may be causative or contributing factors in CRF. Patients with less daytime activity and more nighttime awakenings were noted to consistently report higher levels of CRF. Those with lower peak-activity scores, as measured by wristwatch activity monitors, experienced higher levels of fatigue (Berger et al. Oncol Nurs Forum 26 (10): 1663-71, 1999).

Medications other than chemotherapy may also contribute to fatigue. Opioids used in the treatment of cancer-related pain are often associated with sedation, though the degree of sedation varies among individuals. Opioids are known to alter the normal function of the hypothalamic secretion of gonadotropin-releasing hormone. Other medications—including tricyclic antidepressants, neuroleptics, beta blockers, benzodiazepines, and antihistamines—may produce side effects of sedation. The co-administration of multiple drugs with varying side effects may compound fatigue symptoms.

Since the etiology and mechanisms regarding fatigue/asthenia in cancer patients are indeterminate, there is considerable variation in practice patterns regarding the management of this symptom. The focus of medical management is often directed at identifying specific and potentially reversible correlated symptoms. For example, patients with fatigue and pain may have titration of pain medications; patients with fatigue and anemia may receive a transfusion of packed red blood cells, nutritional interventions including iron-rich foods, supplemental iron or vitamins to correct an underlying deficiency, or injections of epoetin alfa. Patients with depressed mood and fatigue may be treated with antidepressants or psychostimulants. It may also be helpful to consider discontinuation of drugs that may be safely withheld.

There is no agreed-upon approach for the evaluation and treatment of fatigue, but there are an increasing number of clinical trials that are designed to address this issue in cancer patients. Although fatigue is one of the most prevalent symptoms in cancer, to date few trials are published on the use of psychostimulants as a treatment for fatigue in people with cancer. Psychostimulants (caffeine, methylphenidate, modafinil, and dextroamphetamine) given in low doses are useful for patients who are suffering from depressed mood, apathy, decreased energy, poor concentration, and/or weakness. The side effects most commonly associated with psychostimulants include insomnia, euphoria, and mood lability. High doses and long-term use may produce anorexia, nightmares, insomnia, euphoria, paranoia, and possible cardiac complications. The package inserts for all stimulant medications carry boxed warnings indicating risk of abuse potential and/or risk of psychological dependence. Additionally, boxed warnings for certain stimulant medications (methylphenidate and dexmethylphenidate products) indicate risk of psychotic episodes. Other stimulant medications (amphetamine, dextroamphetamine, lisdexamfetamine dimesylate, methamphetamine, and mixed salts of amphetamine products) carry boxed warnings alerting clinicians that misuse of these medications may cause serious cardiovascular adverse events, including sudden death.

In summary, cancer-related fatigue (“CRF”) is a distressing, persistent, subjective sense of physical, emotional and/or cognitive tiredness or exhaustion related to cancer or cancer treatment that is not proportional to recent activity and interferes with usual functioning. Cancer treatment related fatigue (“CTRF”) is a subset of CRF which is diagnosed when all known treatable conditions are ruled out. See Rubenstein, in Pazdur, et al. (eds) Cancer Management: A Multidiscliplinary Approach, 4^{th} ed., PRR, Inc. NY, 2000, pp. 763-770.

The causes of CTRF are complex, difficult to identify, and even more difficult to treat. CTRF is experienced by up to 99% of patients receiving chemotherapy (Schwartz et al. Cancer Invest. 18(1):11-19, 2000. Greater than 30% of patients undergoing treatment report daily fatigue; greater than 20% report fatigue on most days (Fobair, et al. J. Clin. Oncol. 4(5):805-814, 1986). The incidence of CRF among cancer survivors ranges as high as 81%, with 17 to 38% reporting severe fatigue during six months after treatment (Prue, et al. Eur. J. Cancer 42(7):846-863, 2006). 41% of women with stage III breast cancer experience severe fatigue for two to five years post diagnosis. Current treatments, such as psychostimulants, hematopoetic growth factors, antidepressants, complimentary and alternative medicine, activity enhancement, nutrition consultation, and sleep therapy, are mechanistic and not effective.

It is therefore an object of the invention to provide a method for identifying compounds effective to alleviate one or more symptoms of cancer-related fatigue.

It is another object of the present invention to provide compositions for alleviating one or more symptoms of cancer-related fatigue and methods of making and using thereof.

### SUMMARY OF THE INVENTION

Several targets for treatment of CTRF have been identified, including nitrogen metabolism, toll-like receptor signaling, NF-κβ signaling, B cell receptor signaling, P38/MAPK signaling, glutamate receptor signaling, integrin signaling, VEGF signaling, IL-6 signaling, SAPK/JNK signaling, and combinations thereof. Drug classes and/or specific agents that impact these pathways, especially those that impact more than one of these pathways, based on literature and/or laboratory analysis, are identified. In vitro screening is used to identify activity. Animal modeling is then used to confirm activity, optimize dose and formulation, and determine appropriate scheduling of treatment.

An animal model has been developed based on the understanding that a central mechanism in patients with CTRF is that chemotherapy and/or radiation initiates canonical pathways leading to the development of disrupted sleep architecture, resulting in disruption of REM sleep and fatigue and cognitive dysfunction. Mice were treated with etoposide or untreated (controls) and evaluated for the level of activity relative to time of day. Normal animals were active at night. Treated animals became active during the day and had decreased nocturnal activity, as well as a shifted circadian rhythm. Lipopolysaccharide, an inducer of pro-inflammatory cytokines, was used to demonstrate development of fatigue in the model animals. Levels of IL-6 were increased in the treated animals. The results observed in the mice were determined to be independent of anemia.

Drugs that restore the activity patterns and levels towards normal and/or decrease the pro-inflammatory cytokines associated with the disrupted sleep in these model animals should be effective in alleviating one or more symptoms of CTRF. Pentoxifylline was demonstrated to improve activity levels in animals treated with etoposide. Additional drugs, including Armodafinil, methylphenidate, and ALD518 which have similar mechanisms of action, are expected to have benefit in reducing fatigue in this model.

### BRIEF DESCRIPTION OF THE DRAWINGS

FIG. 1 is a graph of total daily activity (counts/group) per day for saline control animals compared to animals treated with lipopolysaccharide (LPS).

FIG. 2 is a graph of total locomotor activity over time (weeks) for animals treated with 60 mg etoposide/kg.

FIG. 3 is a graph of increasing etoposide dosing (0, 50 or 60 mg/kg) causing increased levels of IL-6 (pg/ml).

FIG. 4 is a graph of fatigue (percent of baseline) over time (weeks) for animals treated with 60 mg etoposide/kg, on either a 12 hour dark cycle or a 12 hour light cycle.

FIG. 5 is a graph of the shift of circadian rhythm (measured as percent daily weight change and activity counts) over time in days.

FIG. 6 is a graph showing Pentoxifylline improves activity (total locomotor activity) in animals treated with 60 mg etoposide/kg

FIGS. 7A and 7B are graphs of the effect of Pentoxifylline on 12 hour dark activity (FIG. 7A) as compared to 12 hour light activity (FIG. 7B) over time (weeks) for untreated control, etoposide treated, and etoposide treated followed by Pentoxifylline treated.

### DETAILED DESCRIPTION OF THE INVENTION

### I. Definitions

“Fatigue”, as used herein, refers to a condition marked by extreme tiredness and inability to function due lack of energy. Fatigue may be acute or chronic.

“Acute Symptoms”, as used herein, refers to signs/symptoms that begin and worsen quickly; that is, are not chronic.

“Chronic”, as used herein, refers to a disease or condition that persists or progresses over a long period of time.

“Chronic fatigue syndrome”, as used herein, refers to condition lasting for more than six months in which a person feels tired most of the time and may have trouble concentrating and carrying out daily activities. Other symptoms include sore throat, fever, muscle weakness, headache, and joint pain. Chronic fatigue syndrome is also referred to as CRTF or CFS.

“Pro-inflammatory cytokines” is a general term for those immunoregulatory cytokines that favor inflammation. The major pro-inflammatory cytokines that are responsible for early responses are IL1-alpha, IL1-beta, IL6, and TNF-alpha. Other pro-inflammatory mediators include members of the IL20 family, IL33 LIF, IFN-gamma, OSM, CNTF, TGF-beta, GM-CSF, IL11, IL12, IL17, IL18, IL8 and a variety of other chemokines that chemoattract inflammatory cells. These cytokines either act as endogenous pyrogens (IL1, IL6, TNF-alpha), upregulate the synthesis of secondary mediators and pro-inflammatory cytokines by both macrophages mesenchymal cells (including fibroblasts, epithelial and endothelial cells), stimulate the production of acute phase proteins, or attract inflammatory cells.

### II. Methods of Screening for Compounds to Treat, Alleviate or Prevent CRTF

A. Cellular Assays

In vitro screening assays may also be used to test for drugs that may be effective in treating, alleviating and/or preventing one or more symptoms of CTRF. Several targets for treatment of CTRF have been identified, including nitrogen metabolism, toll-like receptor signaling, NF-κβ signaling, B cell receptor signaling, P38/MAPK signaling, glutamate receptor signaling, integrin signaling, VEGF signaling, IL-6 signaling, and SAPK/JNK signaling. Drug classes and/or specific agents that impact these pathways, especially those that impact more than one of these pathways, based on literature and/or laboratory analysis, are identified. In vitro screening is used to confirm activity. Animal modeling is then used to confirm activity, optimize dose and formulation and determine appropriate scheduling of treatment.

B. Animal Model

An animal model has been developed to screen for drugs which are efficacious in the treatment, alleviation and/or prevention of chronic fatigue syndrome, such as cancer treatment-related fatigue (“CTRF”). The animal model is created by exposing a laboratory animal such as a mouse, rat, guinea pig or rabbit to chemotherapy and/or radiation using a regimen that is comparable to chemotherapy and/or radiation for human cancer patients. The animal's overall activity is measured using a running wheel or other method. In addition, the animal's day and night activity profiles are measured to determine when disrupted sleep architecture is present. This is characterized by disruption of the REM sleep as well as fatigue and cognitive dysfunction. The animal's percent daily weight change is also determined. The levels of cytokines such as IL-6 can be measured, since these are typically elevated with chemotherapy treatment. Results can be provided as percent change of total activity, percent change relative to light exposure, percent weight change over a defined time period (day, week), and combinations thereof.

As demonstrated by the following example using mice treatment with etoposide and etoposide in combination with pentoxifylline, the level of activity relative to time of day (day and night) is indicative of efficacy in alleviating CTRF.

The model can be created using any one of a number of different drugs, alone or in combination with additional therapy, such as one or more other chemotherapeutics or radiation. The in vitro and in vivo models described above can be used to identify compounds that effectively treat CTRF. In one embodiment, etoposide is administered in a dosage of 50 to 60 mg/kg to cause CTRF. In this study etoposide was administered by a single intraperitoneal injection on the first day of the study.

Other compounds used to induce CTRF include cisplatin, BCNU, cytokines such as interferon, arsenic trioxide, taxol and other taxanes, doxorubicin, anti-estrogens or anti-estrogen receptors such as tamoxifen and fulvestrant, testosterone analogs, and/or radiation. These compounds and other chemotherapeutic agents may be administered once or several times, using dosing schedules that recapitulate those used in the clinic.

### III. Compounds to Treat CTRF

As discussed above, several targets for treatment of CTRF have been identified, including nitrogen metabolism, toll-like receptor signaling, NF-κβ signaling, B cell receptor signaling, P38/MAPK signaling, glutamate receptor signaling, integrin signaling, VEGF signaling, IL-6 signaling, and SAPK/JNK signaling.

Toll-like receptors (TLRs) are a class of proteins that play a key role in the innate immune system. They are single, membrane-spanning, non-catalytic receptors that recognize structurally conserved molecules derived from microbes. Once these microbes have breached physical barriers such as the skin or intestinal tract mucosa, they are recognized by TLRs, which activate immune cell responses. Compounds which are known to interact with TLRs include Imiquimod and its successor resiquimod, which have been identified as ligands for TLR7 and TLR8; lipid A analogon eritoran, which acts as a TLR4 antagonist; PF-3512676; and HEPSILAV.

NF-κB (nuclear factor kappa-light-chain-enhancer of activated B cells) is a protein complex that controls the transcription of DNA. NF-κB is found in almost all animal cell types and is involved in cellular responses to stimuli such as stress, cytokines, free radicals, ultraviolet irradiation, oxidized LDL, and bacterial or viral antigens. NF-κB plays a key role in regulating the immune response to infection (kappa light chains are critical components of immunoglobulins). Incorrect regulation of NF-κB has been linked to cancer, inflammatory and autoimmune diseases, septic shock, viral infection, and improper immune development. NF-κB has also been implicated in processes of synaptic plasticity and memory. Drugs which are known to interact with this signaling pathway include denosumab (monoclonal antibody), disulfuram, olmesartan, dithiocarbamates, and anatabine.

The B-cell receptor is a transmembrane receptor protein located on the outer surface of B-cells. The receptor's binding moiety is composed of a membrane-bound antibody that, like all antibodies, has a unique and randomly-determined antigen-binding site. When a B-cell is activated by its first encounter with an antigen that binds to its receptor (its “cognate antigen”), the cell proliferates and differentiates to generate a population of antibody-secreting plasma B cells and memory B cells. Drugs that interact with B-cell receptor signaling include rituximab.

Glutamate receptors are synaptic receptors located primarily on the membranes of neuronal cells. Glutamate is one of the 20 amino acids used to assemble proteins and as a result is abundant in many areas of the body, but it also functions as a neurotransmitter and is particularly abundant in the nervous system. Glutamate receptors are responsible for the glutamate-mediated post-synaptic excitation of neural cells, and are important for neural communication, memory formation, learning, and regulation. Furthermore, glutamate receptors are implicated in the pathologies of a number of neurodegenerative diseases due to their central role in excitotoxicity and their prevalence throughout the central nervous system.

Integrins are receptors that mediate attachment between a cell and the tissues surrounding it, which may be other cells or the ECM. They also play a role in cell signaling and thereby regulate cellular shape, motility, and the cell cycle. Typically, receptors inform a cell of the molecules in its environment and the cell responds. Not only do integrins perform this outside-in signaling, but they also operate an inside-out mode. Thus, they transduce information from the ECM to the cell as well as reveal the status of the cell to the outside, allowing rapid and flexible responses to changes in the environment, for example to allow blood coagulation by platelets.

IL-6 is an interleukin that acts as both a pro-inflammatory and anti-inflammatory cytokine. It is secreted by T cells and macrophages to stimulate immune response, e.g. during infection and after trauma, especially burns or other tissue damage leading to inflammation. Smooth muscle cells in the tunica media of many blood vessels also produce IL-6 as a pro-inflammatory cytokine. IL-6's role as an anti-inflammatory cytokine is mediated through its inhibitory effects on TNF-alpha and IL-1, and activation of IL-1ra and IL-10.

While all compounds that modulate the pathways described above can be used to treat CTRF, specific compounds that may be used here are shown in the following table:

| | | |
|---|---|---|
| Drug | Daily Low Dose | Daily High Dose |

| | | | | |
|---|---|---|---|---|
| Aminophylline | 1 | mg/kg | 10 | mg/kg |
| Paraxanthine | 1 | mg/kg | 20 | mg/kg |
| Pentoxifylline | 300 | mg | 1200 | mg |
| Rolipram | 0.1 | mg/kg | 10 | mg/kg |
| Ibuditant | 1 | mg/kg | 100 | mg/kg |
| Piclamilast | 1 | mg/kg | 10 | mg/kg |
| Luteolin | 10 | mg | 100 | mg |
| Drotaverine | 10 | mg | 50 | mg |
| Sildenafil | 20 | mg | 100 | mg |
| Tadalafil | 5 | mg | 20 | mg |
| Vardenafil | 2.5 | mg | 20 | mg |
| Dipyridamole | 15 | mg | 75 | mg |
| Cilomilast | 5 | mg | 20 | mg |
| Roflumilast | 0.1 | mg | 1 | mg |
| Allopurinol | 100 | mg | 800 | mg |
| Oxypurinol | 20 | mg/kg | 70 | mg/kg |
| Tisopurine | 50 | mg | 150 | mg |
| Febuxostat | 10 | mg | 50 | mg |
| Inositol | 1 | mg/kg | 20 | mg |
| Deslanoside | 0.5 | mg | 1.6 | mg |
| Digitoxin | 0.25 | mg | 1.0 | mg |
| Digoxin | 0.05 | mg | 0.2 | mg |
| Clomipramine | 25 | mg | 250 | mg |
| Imipramine | 10 | mg | 50 | mg |
| Valproate | 250 | mg | 4.5 | g |
| Verapamil | 100 | mg | 500 | mg |
| Desipramine | 50 | mg | 200 | mg |
| Fluvastin | 10 | mg | 50 | mg |
| Lovostatin | 5 | mg | 50 | mg |
| provastatin | 5 | mg | 40 | mg |
| Azalide | 5 | mg | 20 | mg |
| Azithromycin | 100 | mg | 2000 | mg |
| Boromycin | 25 | mg/ml | 500 | mg/ml |
| brefeldin A | 1 | uM | 100 | uM |
| clarithromycin | 10 | mg | 500 | mg |
| dirithromycin | 10 | mg | 500 | mg |
| erythromycin | 10 | mg | 500 | mg |
| fidaxomicin | 10 | mg | 300 | mg |
| flurithromycin | 10 | mg | 500 | mg |
| josamycin | 100 | mg | 1000 | mg |
| kitasamycin | 30 | mg/kg | 400 | mg/kg |
| macrocin | 50 | mg | 500 | mg |
| mepartricin | .001 | mg | .020 | mg |
| midecamycin | 100 | mg | 1200 | mg |
| miocamycin | 0.1 | mg/ml | 1 | mg/ml |
| nargenicin | 10 | mg | 400 | mg/kg |
| oleandomycin | 1 | mg/kg | 100 | mg/kg |
| oligomycin | 1 | mg/ml | 100 | mg/ml |
| pentamycin | 0.5 | mg | 10 | mg/kg |
| pristinamycin | 100 | mg | 500 | mg |
| rokitamycin | 100 | mg | 600 | mg |
| roxithromycin | 50 | mg | 300 | mg |
| solithromycin | 25 | mg | 800 | mg |
| spiramycin | 25 | mg | 100 | mg |
| streptogramin | 100 | mg | 2000 | mg |
| troleandromycin | 200 | mg | 500 | mg |
| tulathromycin | 20 | mg | 100 | mg |
| tylosin | 1 | g | 100 | g |
| virginiamycin | 1 | mg/kg | 5 | mg/kg |
| Chlortetracycline | 50 | mg | 200 | mg |
| Clomocycline | 10 | mg/kg | 50 | mg/kg |
| Demeclocycline | 10 | mg | 500 | mg |
| Doxycline | 5 | mg | 200 | mg |
| Lymecycline | 1 | mg | 500 | mg |
| Meclocycline | 1 | mg | 500 | mg |
| Metacycline | 5 | mg | 400 | mg |
| Minocycline | 1 | mg | 200 | mg |
| Oxytetracycline | 1 | mg | 500 | mg |
| Rolitetracycline | 1 | mg | 500 | mg |
| Tetracycline | 1 | mg | 500 | mg |
| Oxytetracycline | 1 | mg | 500 | mg |
| sulfasalazine | 10 | mg | 500 | mg |
| Leflunomide | 5 | mg | 100 | mg |
| Vincamine | 1 | mg | 200 | mg |
| Vinponcetine | 1 | mg | 100 | mg |
| Tepoxalin | 10 | mg | 400 | mg |

### IV. Compositions for Treatment, Alleviation or Prevention of One or More Symptoms of CTRF

Representative compounds for treatment, alleviation, and/or prevention of one or more symptoms of CTRF include pentoxifylline.

The compounds are typically provided in a pharmaceutically acceptable excipient for administration to an individual in need of treatment thereof. In the preferred embodiment, the formulation is for oral administration, although it may also be administered parenterally, pulmonary or nasal, mucosal (mouth, buccal cavity, vaginal or rectal), or in some cases by transdermal patch or excipient.

The amount of active is that amount effective to alleviate or prevent weight loss, abnormal activity relative to light, lack of sleep, disrupted REM, or to decrease levels of cytokines such as IL-6.

A. Sustained Release Compositions

In one embodiment, the one or more compounds are formulated for sustained or extended release. Sustained or extended release dosage forms provides release of an effective amount of the compound(s) for an extended period of time, such as at least one week, two weeks, three weeks, four weeks, one month, two months, three months, four months, or six months. Sustained or extended release dosage forms can be administered enterally, parenterally, topically, or transdermally.

1. Microparticles and Nanoparticles

In one embodiment, the one or more compounds are formulated as microparticles and/or nanoparticles that provide extended or sustained release of the one or more compounds. In some embodiments, the compounds can be incorporated into a matrix, wherein the matrix provides sustained or extended release. The matrix can contain one or more polymeric and/or non-polymeric materials. In other embodiments, microparticles and/or nanoparticles of drug can be coated with one or more materials that provide sustained or extended release.

Polymers which are slowly soluble in vivo and form a gel in an aqueous environment, such as hydroxypropyl methylcellulose or polyethylene oxide, may be suitable as materials for preparing sustained release drug containing microparticles. Other polymers include, but are not limited to, polyanhydrides, poly(ester anhydrides), polyhydroxy acids, such as polylactide (PLA), polyglycolide (PGA), poly(lactide-co-glycolide) (PLGA), poly-3-hydroxybutyrate (PHB) and copolymers thereof, poly-4-hydroxybutyrate (P4HB) and copolymers thereof, polycaprolactone and copolymers thereof, and combinations thereof.

Alternatively, the one or more compounds can be incorporated into microparticles prepared from materials which are insoluble in aqueous solution or slowly soluble in aqueous solution, but are capable of degrading within the GI tract by means including enzymatic degradation, surfactant action of bile acids, and/or mechanical erosion. As used herein, the term “slowly soluble in water” refers to materials that are not dissolved in water within a period of 30 minutes. Preferred examples include fats, fatty substances, waxes, wax-like substances and mixtures thereof. Suitable fats and fatty substances include fatty alcohols (such as lauryl, myristyl stearyl, cetyl or cetostearyl alcohol), fatty acids and derivatives, including but not limited to fatty acid esters, fatty acid glycerides (mono-, di- and tri-glycerides), and hydrogenated fats. Specific examples include, but are not limited to hydrogenated vegetable oil, hydrogenated cottonseed oil, hydrogenated castor oil, hydrogenated oils available under the trade name Sterotex®, stearic acid, cocoa butter, and stearyl alcohol. Suitable waxes and wax-like materials include natural or synthetic waxes, hydrocarbons, and normal waxes. Specific examples of waxes include beeswax, glycowax, castor wax, carnauba wax, paraffins and candelilla wax. As used herein, a wax-like material is defined as any material which is normally solid at room temperature and has a melting point of from about 30 to 300° C.

In some cases, it may be desirable to alter the rate of water penetration into the microparticles/nanoparticles. To this end, rate-controlling (wicking) agents may be formulated along with the fats or waxes listed above. Examples of rate-controlling materials include certain starch derivatives (e.g., waxy maltodextrin and drum dried corn starch), cellulose derivatives (e.g., hydroxypropylmethyl-cellulose, hydroxypropylcellulose, methylcellulose, and carboxymethyl-cellulose), alginic acid, lactose and talc. Additionally, a pharmaceutically acceptable surfactant (for example, lecithin) may be added to facilitate the degradation of such microparticles.

Proteins which are water insoluble, such as zein, can also be used as materials for the formation of drug containing microparticles. Additionally, proteins, polysaccharides and combinations thereof which are water soluble can be formulated with drug into microparticles and subsequently cross-linked to form an insoluble network. For example, cyclodextrins can be complexed with individual drug molecules and subsequently cross-linked.

Encapsulation or incorporation of drug into carrier materials to produce drug containing microparticles can be achieved through known pharmaceutical formulation techniques. In the case of formulation in fats, waxes or wax-like materials, the carrier material is typically heated above its melting temperature and the drug is added to form a mixture comprising drug particles suspended in the carrier material, drug dissolved in the carrier material, or a mixture thereof. Microparticles can be subsequently formulated through several methods including, but not limited to, the processes of congealing, extrusion, spray chilling or aqueous dispersion. In a preferred process, wax is heated above its melting temperature, drug is added, and the molten wax-drug mixture is congealed under constant stifling as the mixture cools. Alternatively, the molten wax-drug mixture can be extruded and spheronized to form pellets or beads. These processes are known in the art.

For some carrier materials it may be desirable to use a solvent evaporation technique to produce drug containing microparticles. In this case drug and carrier material are co-dissolved in a mutual solvent and microparticles can subsequently be produced by several techniques including, but not limited to, forming an emulsion in water or other appropriate media, spray drying or by evaporating off the solvent from the bulk solution and milling the resulting material.

In some embodiments, drug in a particulate form is homogeneously dispersed in a water-insoluble or slowly water soluble material. To minimize the size of the drug particles within the composition, the drug powder itself may be milled to generate fine particles prior to formulation. The process of jet milling, known in the pharmaceutical art, can be used for this purpose. In some embodiments drug in a particulate form is homogeneously dispersed in a wax or wax like substance by heating the wax or wax like substance above its melting point and adding the drug particles while stifling the mixture. In this case a pharmaceutically acceptable surfactant may be added to the mixture to facilitate the dispersion of the drug particles.

The particles can also be coated with one or more modified release coatings. Solid esters of fatty acids, which are hydrolyzed by lipases, can be spray coated onto microparticles or drug particles. Zein is an example of a naturally water-insoluble protein. It can be coated onto drug containing microparticles or drug particles by spray coating or by wet granulation techniques. In addition to naturally water-insoluble materials, some substrates of digestive enzymes can be treated with cross-linking procedures, resulting in the formation of non-soluble networks. Many methods of cross-linking proteins, initiated by both chemical and physical means, have been reported. One of the most common methods to obtain cross-linking is the use of chemical cross-linking agents. Examples of chemical cross-linking agents include aldehydes (gluteraldehyde and formaldehyde), epoxy compounds, carbodiimides, and genipin. In addition to these cross-linking agents, oxidized and native sugars have been used to cross-link gelatin. Cross-linking can also be accomplished using enzymatic means; for example, transglutaminase has been approved as a GRAS substance for cross-linking seafood products. Finally, cross-linking can be initiated by physical means such as thermal treatment, UV irradiation and gamma irradiation.

To produce a coating layer of cross-linked protein surrounding drug containing microparticles or drug particles, a water soluble protein can be spray coated onto the microparticles and subsequently cross-linked by the one of the methods described above. Alternatively, drug containing microparticles can be microencapsulated within protein by coacervation-phase separation (for example, by the addition of salts) and subsequently cross-linked. Some suitable proteins for this purpose include gelatin, albumin, casein, and gluten.

Polysaccharides can also be cross-linked to form a water-insoluble network. For many polysaccharides, this can be accomplished by reaction with calcium salts or multivalent cations which cross-link the main polymer chains. Pectin, alginate, dextran, amylose and guar gum are subject to cross-linking in the presence of multivalent cations. Complexes between oppositely charged polysaccharides can also be formed; pectin and chitosan, for example, can be complexed via electrostatic interactions.

i. Enteral Formulations

The microparticles and/or nanoparticles can be formulated for enteral administration. Suitable dosage forms include, but are not limited to, tablets, caplets, hard and soft capsules (e.g., gelatin or non-gelatin capsules), and suspensions. In some embodiments, the one or more compounds are incorporated into a sustained or extended release matrix and the matrix is formulated into a suitable dosage form. For example, particles of the compounds incorporated into the matrix can be pressed into tablet, encapsulated in a hard or soft capsule, or suspended in a solvent. In other embodiments, microparticles or nanoparticles of the one or more compounds can be coated with one or more materials that provide sustained or extended release and the coated particles can be formulated into an oral dosage form, such as a tablet or capsule. The dosage form itself can also be coated with one or more coating materials to delay release until the dosage form passes through the stomach and/or one or more materials which provide sustained or extended release.

Formulations may be prepared using a pharmaceutically acceptable carrier. As generally used herein “carrier” includes, but is not limited to, diluents, preservatives, binders, lubricants, disintegrators, swelling agents, fillers, stabilizers, and combinations thereof.

Carrier also includes all components of the coating composition which may include plasticizers, pigments, colorants, stabilizing agents, and glidants. Delayed release dosage formulations may be prepared as described in standard references. These references provide information on carriers, materials, equipment and process for preparing tablets and capsules and delayed release dosage forms of tablets, capsules, and granules.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

Optional pharmaceutically acceptable excipients include, but are not limited to, diluents, binders, lubricants, disintegrants, colorants, stabilizers, and surfactants. Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone® XL from GAF Chemical Corp).

Stabilizers are used to inhibit or retard drug decomposition reactions which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

ii. Parenteral Formulations

The microparticles and/or nanoparticles can be formulated for parenteral administration. “Parenteral administration”, as used herein, means administration by any method other than through the digestive tract or non-invasive topical or regional routes. For example, parenteral administration may include administration to a patient intravenously, intradermally, intraarterially, intraperitoneally, intralesionally, intracranially, intraarticularly, intraprostatically, intrapleurally, intratracheally, intravitreally, intratumorally, intramuscularly, subcutaneously, subconjunctivally, intravesicularly, intrapericardially, intraumbilically, by injection, and by infusion.

Parenteral formulations can be prepared as aqueous compositions using techniques is known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, microemulsions, liposomes, or emulsomes.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the active compounds as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, viscosity modifying agents, and combination thereof.

Suitable surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s).

The formulation is typically buffered to a pH of 3-8 for parenteral administration upon reconstitution. Suitable buffers include, but are not limited to, phosphate buffers, acetate buffers, and citrate buffers.

Water soluble polymers are often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the active compounds in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The powders can be prepared in such a manner that the particles are porous in nature, which can increase dissolution of the particles. Methods for making porous particles are well known in the art.

2. Injectable/Implantable Solid Implants

The one or more compounds described herein can be incorporated into injectable/implantable solid or semi-solid implants, such as polymeric implants. In one embodiment, the one or more compounds are incorporated into a polymer that is a liquid or paste at room temperature, but upon contact with aqueous medium, such as physiological fluids, exhibits an increase in viscosity to form a semi-solid or solid material. Exemplary polymers include, but are not limited to, hydroxyalkanoic acid polyesters derived from the copolymerization of at least one unsaturated hydroxy fatty acid copolymerized with hydroxyalkanoic acids. The polymer can be melted, mixed with the active substance and cast or injection molded into a device. Such melt fabrication requires polymers having a melting point that is below the temperature at which the substance to be delivered and polymer degrade or become reactive. The device can also be prepared by solvent casting where the polymer is dissolved in a solvent and the drug dissolved or dispersed in the polymer solution and the solvent is then evaporated. Solvent processes require that the polymer be soluble in organic solvents. Another method is compression molding of a mixed powder of the polymer and the drug or polymer particles loaded with the active agent. ATRIGEL® is another example of a formulation which forms a solid implant in situ upon contact with aqueous fluids.

Alternatively, the one or more compounds described herein can be incorporated into a polymer matrix and molded, compressed, or extruded into a device that is a solid at room temperature. For example, the one or more compounds can be incorporated into a biodegradable polymer, such as polyanhydrides, polyhydroalkanoic acids (PHAs), PLA, PGA, PLGA, polycaprolactone, polyesters, polyamides, polyorthoesters, polyphosphazenes, proteins and polysaccharides such as collagen, hyaluronic acid, albumin and gelatin, and combinations thereof and compressed into solid device, such as disks, or extruded into a device, such as rods.

In other embodiments, the solid implant is in the form of a silastic implant.

The release of the one or more compounds from the implant can be varied by selection of the polymer, the molecular weight of the polymer, and/or modification of the polymer to increase degradation, such as the formation of pores and/or incorporation of hydrolyzable linkages. Methods for modifying the properties of biodegradable polymers to vary the release profile of the one or more compounds from the implant are well known in the art.

3. Topical Formulations

The one or more compounds can be administered topically. Suitable dosage forms for topical administration include creams, ointments, salves, sprays, gels, lotions, emulsions, and transdermal patches. The formulation may be formulated for transmucosal, transepithelial, transendothelial, or transdermal administration. The compositions may contain one or more excipients suitable for topical administration, such as chemical penetration enhancers, membrane permeability agents, membrane transport agents, emollients, surfactants, stabilizers, and combinations thereof.

“Emollients” are an externally applied agent that softens or soothes skin and are generally known in the art and listed in compendia, such as the “Handbook of Pharmaceutical Excipients”, 4^{th} Ed., Pharmaceutical Press, 2003. These include, without limitation, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In one embodiment, the emollients are ethylhexylstearate and ethylhexyl palmitate.

“Surfactants” are surface-active agents that lower surface tension and thereby increase the emulsifying, foaming, dispersing, spreading and wetting properties of a product. Suitable non-ionic surfactants include emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In one embodiment, the non-ionic surfactant is stearyl alcohol.

“Emulsifiers” are surface active substances which promote the suspension of one liquid in another and promote the formation of a stable mixture, or emulsion, of oil and water. Common emulsifiers are: metallic soaps, certain animal and vegetable oils, and various polar compounds. Suitable emulsifiers include acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In one embodiment, the emulsifier is glycerol stearate.

Suitable classes of penetration enhancers are known in the art and include, but are not limited to, fatty alcohols, fatty acid esters, fatty acids, fatty alcohol ethers, amino acids, phospholipids, lecithins, cholate salts, enzymes, amines and amides, complexing agents (liposomes, cyclodextrins, modified celluloses, and diimides), macrocyclics, such as macrocylic lactones, ketones, and anhydrides and cyclic ureas, surfactants, N-methylpyrrolidones and derivatives thereof, DMSO and related compounds, ionic compounds, azone and related compounds, and solvents, such as alcohols, ketones, amides, polyols (e.g., glycols). Examples of these classes are known in the art.

i. Lotions, Creams, Gels, Ointments, Emulsions, and Foams

In some embodiments, the compounds can be applied topically in the form of a lotion, cream, gel, ointment, emulsion, or foam. These dosage forms typically contain hydrophilic and hydrophobic materials, for example, to form an emulsion.

“Hydrophilic” as used herein refers to substances that have strongly polar groups that readily interact with water.

“Lipophilic” refers to compounds having an affinity for lipids.

“Amphiphilic” refers to a molecule combining hydrophilic and lipophilic (hydrophobic) properties

“Hydrophobic” as used herein refers to substances that lack an affinity for water; tending to repel and not absorb water as well as not dissolve in or mix with water.

A “gel” is a colloid in which the dispersed phase has combined with the continuous phase to produce a semisolid material, such as jelly.

An “oil” is a composition containing at least 95% wt of a lipophilic substance. Examples of lipophilic substances include but are not limited to naturally occurring and synthetic oils, fats, fatty acids, lecithins, triglycerides and combinations thereof.

A “continuous phase” refers to the liquid in which solids are suspended or droplets of another liquid are dispersed, and is sometimes called the external phase. This also refers to the fluid phase of a colloid within which solid or fluid particles are distributed. If the continuous phase is water (or another hydrophilic solvent), water-soluble or hydrophilic drugs will dissolve in the continuous phase (as opposed to being dispersed). In a multiphase formulation (e.g., an emulsion), the discreet phase is suspended or dispersed in the continuous phase.

An “emulsion” is a composition containing a mixture of non-miscible components homogenously blended together. In particular embodiments, the non-miscible components include a lipophilic component and an aqueous component. An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. The oil phase may consist at least in part of a propellant, such as an HFA propellant. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

A sub-set of emulsions are the self-emulsifying systems. These drug delivery systems are typically capsules (hard shell or soft shell) comprised of the drug dispersed or dissolved in a mixture of surfactant(s) and lipophilic liquids such as oils or other water immiscible liquids. When the capsule is exposed to an aqueous environment and the outer gelatin shell dissolves, contact between the aqueous medium and the capsule contents instantly generates very small emulsion droplets. These typically are in the size range of micelles or nanoparticles. No mixing force is required to generate the emulsion as is typically the case in emulsion formulation processes.

A “lotion” is a low- to medium-viscosity liquid formulation. A lotion can contain finely powdered substances that are in soluble in the dispersion medium through the use of suspending agents and dispersing agents. Alternatively, lotions can have as the dispersed phase liquid substances that are immiscible with the vehicle and are usually dispersed by means of emulsifying agents or other suitable stabilizers. In one embodiment, the lotion is in the form of an emulsion having a viscosity of between 100 and 1000 centistokes. The fluidity of lotions permits rapid and uniform application over a wide surface area. Lotions are typically intended to dry on the skin leaving a thin coat of their medicinal components on the skin's surface.

A “cream” is a viscous liquid or semi-solid emulsion of either the “oil-in-water” or “water-in-oil type”. Creams may contain emulsifying agents and/or other stabilizing agents. In one embodiment, the formulation is in the form of a cream having a viscosity of greater than 1000 centistokes, typically in the range of 20,000-50,000 centistokes. Creams are often time preferred over ointments as they are generally easier to spread and easier to remove.

The difference between a cream and a lotion is the viscosity, which is dependent on the amount/use of various oils and the percentage of water used to prepare the formulations. Creams are typically thicker than lotions, may have various uses and often one uses more varied oils/butters, depending upon the desired effect upon the skin. In a cream formulation, the water-base percentage is about 60-75% and the oil-base is about 20-30% of the total, with the other percentages being the emulsifier agent, preservatives and additives for a total of 100%.

An “ointment” is a semisolid preparation containing an ointment base and optionally one or more active agents. Examples of suitable ointment bases include hydrocarbon bases (e.g., petrolatum, white petrolatum, yellow ointment, and mineral oil); absorption bases (hydrophilic petrolatum, anhydrous lanolin, lanolin, and cold cream); water-removable bases (e.g., hydrophilic ointment), and water-soluble bases (e.g., polyethylene glycol ointments). Pastes typically differ from ointments in that they contain a larger percentage of solids. Pastes are typically more absorptive and less greasy that ointments prepared with the same components.

A “gel” is a semisolid system containing dispersions of small or large molecules in a liquid vehicle that is rendered semisolid by the action of a thickening agent or polymeric material dissolved or suspended in the liquid vehicle. The liquid may include a lipophilic component, an aqueous component or both. Some emulsions may be gels or otherwise include a gel component. Some gels, however, are not emulsions because they do not contain a homogenized blend of immiscible components. Suitable gelling agents include, but are not limited to, modified celluloses, such as hydroxypropyl cellulose and hydroxyethyl cellulose; Carbopol homopolymers and copolymers; and combinations thereof. Suitable solvents in the liquid vehicle include, but are not limited to, diglycol monoethyl ether; alklene glycols, such as propylene glycol; dimethyl isosorbide; alcohols, such as isopropyl alcohol and ethanol. The solvents are typically selected for their ability to dissolve the drug. Other additives, which improve the skin feel and/or emolliency of the formulation, may also be incorporated. Examples of such additives include, but are not limited, isopropyl myristate, ethyl acetate, C₁₂-C₁₅ alkyl benzoates, mineral oil, squalane, cyclomethicone, capric/caprylic triglycerides, and combinations thereof.

Foams consist of an emulsion in combination with a gaseous propellant. The gaseous propellant consists primarily of hydrofluoroalkanes (HFAs). Suitable propellants include HFAs such as 1,1,1,2-tetrafluoroethane (HFA 134a) and 1,1,1,2,3,3,3-heptafluoropropane (HFA 227), but mixtures and admixtures of these and other HFAs that are currently approved or may become approved for medical use are suitable. The propellants preferably are not hydrocarbon propellant gases which can produce flammable or explosive vapors during spraying. Furthermore, the compositions preferably contain no volatile alcohols, which can produce flammable or explosive vapors during use.

Buffers are used to control pH of a composition. Preferably, the buffers buffer the composition from a pH of about 4 to a pH of about 7.5, more preferably from a pH of about 4 to a pH of about 7, and most preferably from a pH of about 5 to a pH of about 7. In a preferred embodiment, the buffer is triethanolamine.

Preservatives can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal.

ii. Patches

For topical applications, repeated application can be done or a patch can be used to provide continuous administration of the compounds over an extended period of time.

iii. Implants

Implants can be used to provide sustained delivery. In one embodiment, the implant is the Alza minipump; in another it is an insulin type pump; in still another embodiment, it is a silastic tube of the type used to deliver birth control hormones, such as IMPLANON®.

### V. Methods of Treatment

Compounds are typically administered with or immediately after administration of the chemotherapy and/or radiation, in an amount and regimen to treat, alleviate or prevent one or more symptoms of CTRF. However, administration can begin at any point following development of CTRF. For example, in some embodiments, the one or more compounds are administered every day during the course of chemotherapy and/or radiation treatment and then daily or less than daily for a period of time after the chemotherapy and/or radiation, such as a week, two weeks, four weeks, one month, two months, three months, four months, six months, one year, 18 months, or two years.

The present invention will be further understood by reference to the following non-limiting examples.

### Example 1

### Development of Animal Model for CTRF

Materials and Methods

For the LPS-induced fatigue model shown in FIG. 1, thirty, female BALB/c mice were obtained from Charles River Laboratories (Wilmington, Mass.). The mice were randomized into 2 groups of 10 prior to treatment. Animals were housed 5 per cage in micro-isolators and allowed to acclimatize for 4 days prior to dosing. Animal were given food and water ad libitum with a 12 hour light/12 hour dark schedule.

Animals in the control group were initially injected intraperitoneally with saline. Animals in the LPS group were injected intraperitoneally with a single dose of 2.5 mg/kg lipopolysaccharide on dayl. This treatment induced pro-inflammatory cytokines and causes CTRF. Daily activity counts were taken and the decreased activity induced by LPS is shown in FIG. 1.

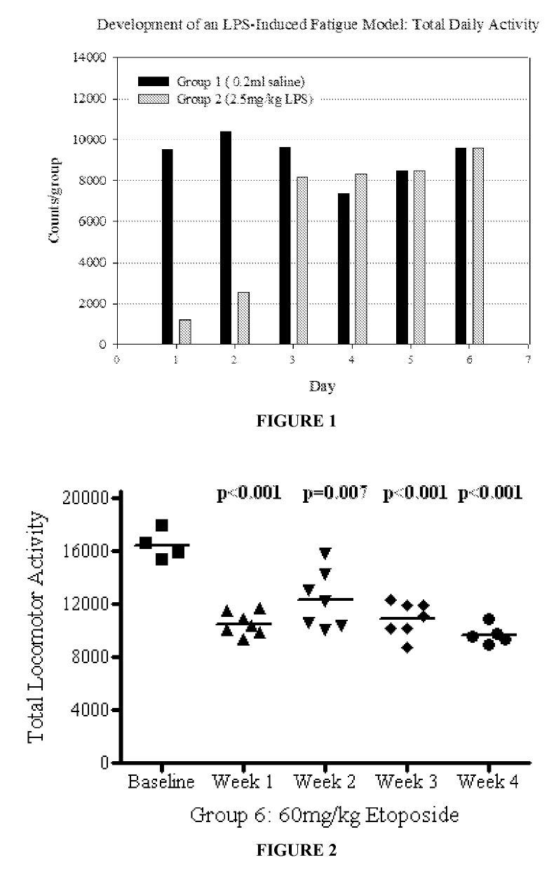

For the etoposide-induced fatigue model (FIGS. 2-7), BALB/c mice (obtained and housed as described above) were divided into groups of 10 animals per group: 1. control (untreated) mice, 2. mice treated with a single intraperitoneal dose of 60 mg/kg etoposide on day 1, and 3. mice treated with a single intraperitoneal dose of 60 mg/kg etoposide on day 1, followed by daily oral doses of pentoxifylline administered as a 1 mg/kg dose in the drinking water throughout the study. The animals were then assessed for their level of activity relative to time of day (day and night); total locomotor activity; blood chemistry (hemoglobin, red blood cells, white blood cells); over time in weeks.

Results

FIG. 1 is a graph of total daily activity (counts/group) per day for saline control animals compared to animals treated with LPS. The results indicate that the LPS caused decreased activity indicative of fatigue.

FIG. 2 is a graph of total locomotor activity over time (weeks) for animals treated with 60 mg etoposide/kg. The results are consistent with those for FIG. 1, showing that etoposide also causes CTRF, with decreased total activity over time of treatment.

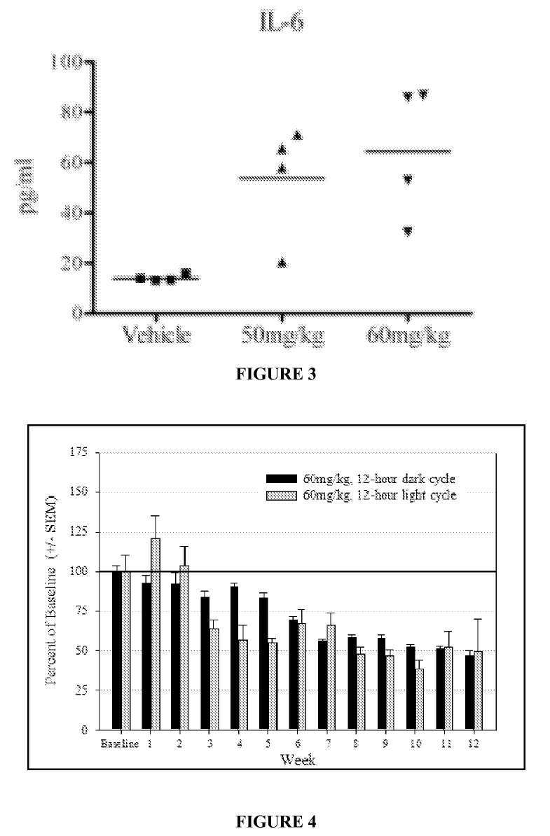

FIG. 3 is a graph of increasing etoposide dosing (0, 50 or 60 mg/kg) causing increased levels of IL-6 (pg/ml), an indication that the etoposide is causing increased levels of pro-inflammatory cytokine release.

FIG. 4 is a graph of fatigue (percent of baseline) over time (weeks) for animals treated with 60 mg etoposide/kg, on either a 12 hour dark cycle or a 12 hour light cycle. The results indicate that there is decreased activity for animals treated with etoposide both during the day and night.

FIG. 5 is a graph of the shift of circadian rhythm (measured as percent daily weight change and activity counts) over time in days. The results show that etoposide caused weight loss and decreased activity, which was more pronounced during the evening, the time the animals are normally most active.

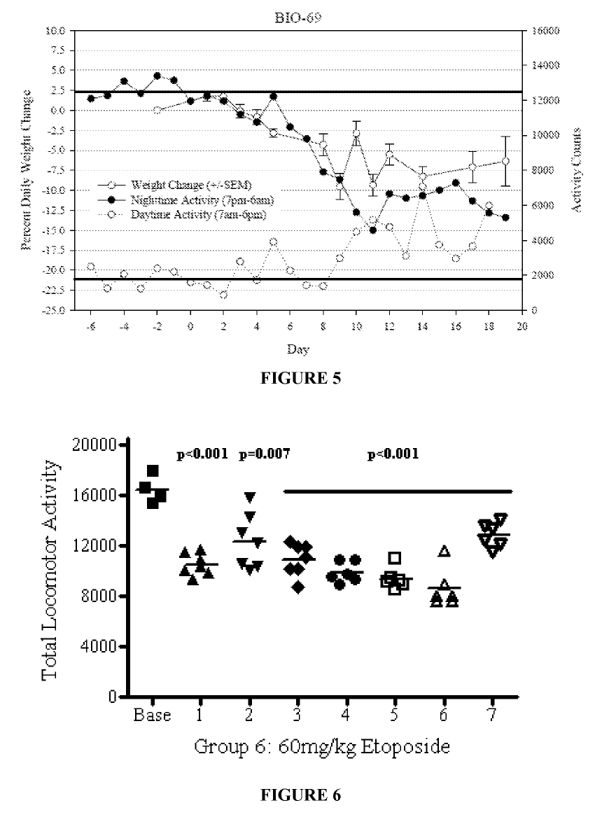

FIG. 6 is a graph showing Pentoxifylline improves activity (total locomotor activity) in animals treated with 60 mg etoposide/kg. This indicates that pentoxifylline can reverse some of the negative effects of the etoposide on activity levels.

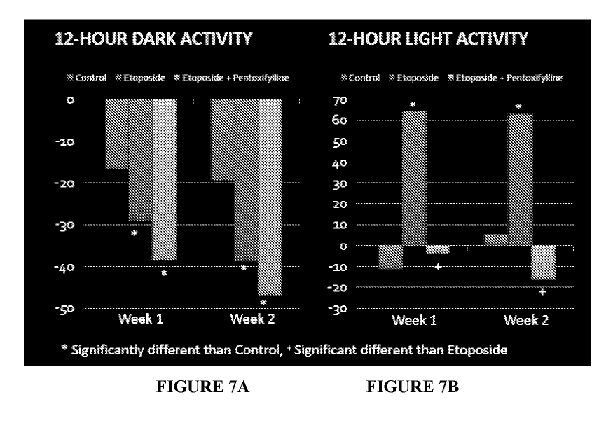

FIGS. 7A and 7B are graphs of the effect of Pentoxifylline on 12 hour dark activity (FIG. 7A) as compared to 12 hour light activity (FIG. 7B) over time (weeks) for untreated control, etoposide treated, and etoposide treated followed by Pentoxifylline treated. The results demonstrate that pentoxifylline can significantly increase nocturnal activity, but also decrease activity when animals should be sleeping (i.e., during the light cycle), which are not only related to fatigue but are indicators of restoration of normal sleep and circadian rhythms).

In summary, the results validate the animal model and the use of pentoxifylline to treat, prevent and/or alleviate one or more symptoms of CTRF induced by chemotherapy and/or radiation.

Modifications and variations will be apparent to those skilled in the art and are intended to come within the scope of the appended claims. References cited herein are specifically incorporated herein.

We claim:

1. A method of alleviating cancer treatment-related fatigue comprising administering an effective amount of a drug which restores the activity/sleep patterns and levels towards normal and/or decreases the pro-inflammatory cytokines associated with disrupted sleep.

2. The method of claim 1 wherein the drug increases IL-6 levels.

3. The method of claim 1 wherein the pro-inflammatory cytokines are TNF-alpha, IL-1, or an activator of IL-1ra and IL-10.

4. The method of claim 1 wherein the cancer treatment is chemotherapy, radiation or a combination thereof.

5. The method of claim 1, wherein the drug is selected from the group consisting of Aminophylline, Paraxanthine, Pentoxifylline, Rolipram, Ibuditant, Piclamilast, Luteolin, Drotaverine, Sildenafil, Tadalafil, Vardenafil, Dipyridamole, Cilomilast, Roflumilast, Allopurinol, Oxypurinol, Tisopurine, Febuxostat, Inositol, Deslanoside, Digitoxin, Digoxin, Clomipramine, Imipramine, Valproate, Verapamil, Desipramine, Fluvastin, Lovostatin, pravastatin, Azalide,
Azithromycin, Boromycin, brefeldin A, clarithromycin, dirithromycin, erythromycin, fidaxomicin, flurithromycin, josamycin, kitasamycin, macrocin Mepartricin, midecamycin, miocamycin, nargenicin, oleandomycin, oligomycin, Pentamycin, pristinamycin, rokitamycin, roxithromycin, solithromycin, spiramycin, streptogramin, troleandromycin, tulathromycin, tylosin, virginiamycin, Chlortetracycline, Clomocycline, Demeclocycline, Doxycline, Lymecycline, Meclocycline, Metacycline, Minocycline, Oxytetracycline, Rolitetracycline, Tetracycline, Oxytetracycline, sulfasalazine, Leflunomide, Vincamine, Vinponcetine, Tepoxalin, and combinations thereof.

6. The method of claim 1 wherein the drug is Pentoxifylline, Armodafinil, methylphenidate, or ALD518.

7. The method of claim 1 wherein the drug is provided in a sustained release formulation or implant.

8. A formulation for use in the method of claim 1.

9. The formulation of claim 8 comprising Pentoxifylline.

10. The formulation of claim 8 wherein the drug is selected from the group consisting of Aminophylline, Paraxanthine, Pentoxifylline, Rolipram, Ibuditant, Piclamilast, Luteolin, Drotaverine, Sildenafil, Tadalafil, Vardenafil, Dipyridamole, Cilomilast, Roflumilast, Allopurinol, Oxypurinol, Tisopurine, Febuxostat, Inositol, Deslanoside, Digitoxin, Digoxin, Clomipramine, Imipramine, Valproate, Verapamil, Desipramine, Fluvastin, Lovostatin, pravastatin, Azalide, Azithromycin, Boromycin, brefeldin A, clarithromycin, dirithromycin, erythromycin, fidaxomicin, flurithromycin, josamycin, kitasamycin, macrocin, Mepartricin, midecamycin, miocamycin, nargenicin, oleandomycin, oligomycin, Pentamycin, pristinamycin, rokitamycin, roxithromycin, solithromycin, spiramycin, streptogramin, troleandromycin, tulathromycin, tylosin, virginiamycin, Chlortetracycline, Clomocycline, Demeclocycline, Doxycline, Lymecycline, Meclocycline, Metacycline, Minocycline, Oxytetracycline, Rolitetracycline, Tetracycline, Oxytetracycline, sulfasalazine, Leflunomide, Vincamine, Vinponcetine, Tepoxalin, and combinations thereof.
